(19) United States
(12) Patent Application Publication
(10) Pub. No.: US 2007/0292912 A1
(43) Pub. Date: Dec. 20, 2007

(54) Diffusion-controlling sensor layer

(75) Inventors: Wolfgang Kreiss, Bergisch Gladbach (DE); Gunther Eberz, Leverkusen (DE); Claus Weisemann, Apex, NC (US); Klaus Burger, Neuss (DE)

Correspondence Address: EDWARDS ANGELL PALMER & DODGE LLP, P.O. BOX 55874, BOSTON, MA, 02205, US

(73) Assignee: Bayer Aktiengesellschaft

(21) Appl. No.: 11/893,800

(22) Filed: Aug. 16, 2007

Related U.S. Application Data
(62) Division of application No. 09/527,767, filed on Mar. 17, 2000, now abandoned.

(30) Foreign Application Priority Data
Apr. 3, 1999 (DE) 19915310.8

(51) Int. Cl. C12Q 1/68

(52) U.S. Cl. 435/29

(57) ABSTRACT

The invention relates to a diffusion-controlling sensor layer for detecting the biological effect of substances consisting of a diffusion-controlling matrix and sensors suspended therein. The invention further comprises a method and an apparatus for the location-dependent detection of the biological effect of substances using the sensor layer according to the invention (“imaging” of the spatial distribution of biological activity). A further embodiment of the method according to the invention relates to the coupling of investigations of the biological activity of substances with chromatographic and electrophoretic separation techniques so that biologically active individual substances can be identified directly in mixtures. An additional embodiment of the method according to the invention comprises the direct combination of the detection of the effect via sensor layers with chromatographic and electrophoretic separation techniques which are in turn coupled to spectroscopic techniques. This close coupling of substance separation, detection of effect and spectroscopy directly provides results on the structure of the biologically active substances in a mixture of substances.

### CROSS-REFERENCE TO RELATED APPLICATIONS

This application is a divisional application of U.S. patent application Ser. No. 09/527,767, filed Mar. 17, 2000, which application is hereby incorporated by reference in its entirety.

Information on the biological effect of substances is essential for the development and use of active substances, especially in the pharmaceutical sector. Tests of effects represent crucial steps in the assessment of the results of combinatorial chemistry and the evaluation of natural substances or of synthetic substance libraries. The invention relates to a diffusion-controlling sensor layer, an apparatus and a method for detecting the biological effect of substances.

Generally used for testing biological effects are microtitre plate formats or formats derived therefrom. A common feature of these techniques is that the assay of effect is carried out in separate compartments (microtitre plate well, vial, sensor point in sensor array). In these cases, the substance to be tested is brought into contact in discrete liquid volumes, for example inside the microtitre plate wells, with the sensor system. The biological effect is detected in the individual microtitre plate wells via the particular reaction of the sensor system, for example change in colour in the presence of bioactive substances [High Throughput Screening, John P. Devlin, Marcel Dekker INC, New York, 1997]. In the state of the art, the sensor organisms are present in a suspension in which they are able to diffuse freely. In a suspension there may be sedimentation of the sensor organisms during the detection process. In addition, no uniform coating of different materials (glass, plastic, metal) and surfaces (smooth, rough, porous) is achieved.

Although the test methods corresponding to the state of the art provide information on the effect of a sample as a whole, because they are carried out discontinuously they are unable to image the spatial distribution of biological activity on the surface of objects under investigation.

A further restriction on assay methods according to the state of the art comprises the sensitivity to interference leading to false-negative results. Thus, cytotoxic substances interfere with conventional cell-based assays, while enzymatic assays are influenced, for example, by denaturing substances. These interfering components may be present as contamination in the sample to be tested or are a constituent of the substance mixtures, as is frequently the case with natural substances.

With the established tests of effects there is the problem of adjusting under the measurement conditions the optimal concentrations or activities of the substances to be tested. Under real assay conditions, the requirements resulting therefrom for sample preparation for substances, some of which are unknown, can be complied with only at great expense.

Assays of effects which are based on a multistage process (for example based on β-galactosidase expression with a subsequent colour reaction) require in the state of the art a multistep procedure with the consequent increased expense for carrying out the assay.

Samples suitable for tests of effects by known methods are pure substances. However, substances are usually present in mixtures in practice.

In this regard, EP 588 139 describes how the biological effect of substances is examined by a combination of chromatographic fractionation of the substances to be assayed into chromatographic zones with a subsequent assay of the biological effect (the toxicity) of the individual fractions. This entails the individual fractions being brought into contact with luminescent microorganisms which indicate the biological effect of a fraction by a local change in their bioluminescence on the individual fractions.

The separation of the substances into fractions takes place, for example, by thin-layer chromatography (TLC) or column chromatography (HPLC). In the case of thin-layer chromatography, the TLC plate is wetted with a luminescent microorganism suspension, and the local bioluminescence assigned to the individual fractions is investigated. In the case of separation by column chromatography, the luminescent microorganism suspension is continuously admixed to the eluate from the chromatography column, and the bioluminescence of the mixture is measured.

P. D. Shaw et al., Proc. Natl. Acad. Sci. USA, Vol. 94, 1997, 6036-6041 describe a thin-layer chromatography method in which the detection of homoserine lactone takes place via a reporter gene system in a 3 mm-thick agar layer as nutrient medium. This method is employed exclusively for the analytical detection of homoserine lactone. The detection system described for detecting homoserine lactone is based on a modified Agrobacterium tumefaciens and dye formation. It is possible with the large layer thickness described herein to achieve only a relatively poor imaging performance and high detection limits.

WO 97/16569 describes detection of the effect of substances which are obtained exclusively by solid-phase synthesis, for example on polymer beads. Enzymatic assays and binding assays are mentioned for detecting the effect. There is no mention of a general application for test substances of any origin. Nor is it possible with the method described in WO 97/16569 to achieve the options, which are important for practical use, of direct coupling with chromatographic separation techniques or with spectroscopic structure-elucidation methods.

WO 94/02515 likewise discloses the liberation of substances from polymer beads and a subsequent detection of the effect (cell-based assays). Once again, more general applications are not mentioned.

EP 063 810 A1 describes a specific embodiment of immunoassays for diagnostic applications. The application is aimed at the production and use of test strips for tests of medical relevance.

The object of the invention is to find a possible way of detecting the biological effect of substances which is, compared with the state of the art, simpler, more sensitive, faster, more widely applicable and associated with a smaller risk of artefacts.

The object according to the invention is achieved by a sensor layer for detecting the biological effect of substances, which is brought into contact with the sample, consisting of a diffusion-controlling matrix and sensors suspended therein.

The matrix can in this case be a secondary valence gel, for example agarose, a polymer gel, for example acrylate or a viscous solution, for example polyethylene glycol in water. Agar is less suitable because of the relatively high gel point, in particular in thermolabile sensors.

Sensor systems suitable in principle are all sensors which are suitable for tests of effects and which can be incorporated into sensor layers. Examples of suitable sensors are microorganisms, in particular cells with reporter gene constructs, enzyme systems, antibodies and fluorescent sensors.

The sensor layer forms a diffusion barrier for substances in contact with it. Different substances diffuse at different rates and to different extents into the sensor layer because mass transport in the sensor matrix takes place substance-specifically, inter alia dependent on polarity and molecule size. This results in the formation of a concentration gradient within the sensor layer for the individual fractions or substances to be tested. An optimal concentration of the substance or fraction to be tested is present at a particular distance from the carrier for each detection principle with the relevant sensor. The concentration-dependent effect can also be observed within the profile. In addition, interfering substances such as impurities are separated by the diffusion process spatially within the sensor layer from active substances.

It is furthermore possible to introduce into the sensor layer additions which directly control the detection process by influencing the detection sensitivity, the selectivity and the kinetics of the sensor layer. An example of an addition of this type is a buffer for regulating the vitality status of sensor cells.

The diffusion-controlling sensor layer can also take up indicator substances (for example pH indicators, redox dyes) which make it possible to obtain spatially resolving information on pH values or redox properties on surfaces of objects under investigation.

The addition of bioluminescent substrates, chemiluminescent reagents, fluorescent reagents and other components which play a part in particular test methods to the sensor layer makes it possible to carry out multistage detection processes in the sensor layer in one operation. An example of such a multistage reaction is β-galactosidase expression with subsequent dye formation or fluorescent or chemiluminescent reaction.

The thickness of the sensor layer is preferably 0.1 to 10 mm, particularly preferably 0.5 to 3 mm, very particularly preferably 0.5 to 0.8 mm.

In a preferred embodiment, the sensor layer contains 2 to 8 ml of reporter gene cell suspension, particularly preferably 3 to 5 ml of reporter gene cell suspension, in 50 ml of sensor layer composition.

The reporter gene cell suspension employed preferably has an optical density of 0.6 to 1.4 at a wavelength of 660 nm.

The sensor layer itself may be constructed from a plurality of layers. In this case, the individual layers serve a variety of purposes and may differ in thickness appropriate for their purpose. The individual layers may have the following function:
uptake of different sensor systems for multiple detection
uptake of additions to control and assist the detection process
effect as diffusion barrier or substance-selective filter layer.

It is also possible for a plurality of different sensors differing in specificity for effects to be suspended in the matrix for simultaneous detection of different biological effects. This results in a multisensor layer. In order to distinguish which sensor in such a multisensor layer shows a biological effect it is necessary for the signals emitted by the individual sensors to be different. Examples thereof are a multisensor layer with a plurality of reporter gene cell lines which indicate different biological activities with different signals such as, for example, bioluminescence, GFP fluorescence, β-galactosidase expression. Instead of a plurality of different sensors it is also possible to use reporter gene cell lines which indicate a plurality of different effects simultaneously. The signals from the multisensor layer can be analysed in parallel.

The object according to the invention is additionally achieved by a method for testing the biological activity of substances. The sample is initially put onto or into the surface of a carrier unless it is already a constituent of a carrier. The carrier can be either the sensor layer itself or an additional carrier. If the sensor layer does not itself serve as carrier, the carrier is then covered with the sensor layer according to the invention. The effect of the substance on the sensors in the sensor layer is then determined.

Carriers which can be used, apart from the sensor layer itself, are smooth, structured or porous objects made of glass, plastics, metal or of other organic or inorganic materials. Paper, membranes, films, sheets or polymer beads are particularly suitable. It is also possible for biological material such as tissue sections or plant leaves to serve directly as sample so that, in this case, the sample is a constituent of the carrier. The sensor layer can be applied to the carrier by casting, dipping, rolling, spraying or as film.

If the sensor layer itself serves as carrier, the substance to be assayed is applied directly to the sensor layer, for example by micrometering systems or printing techniques in which substances are transferred to the layer. A printing technique consists, for example, of immersing the needles of a plunger into the substances to be assayed, which are, for example, in the wells of a microtitre plate. This results in each individual substance wetting a needle tip. The plunger with the needles is then pushed into the sensor layer. An alternative possibility is also to apply the sample substances to paper and press this paper with the side coated by the sample substances onto the sensor layer.

On use of a sensor layer which contains cells as sensors, an incubation step precedes the determination of the effect of the substances present in the sample on the sensors. For this purpose, the sensor layer or the carrier covered with the sensor layer is stored in accordance with the requirements of the cells employed under defined conditions in relation to temperature, humidity and gas introduction for a preset time. Only then is the effect of the substance to be assayed on the sensors determined.

The method according to the invention makes it possible for concentration methods to be combined directly with the assay of effect. Concentrations are necessary, for example, if the substances have low activity. The constituents of a sample solution can be concentrated by nonspecific or specific adsorption onto suitable carriers such as membranes, ion exchange matrices, affinity matrices, thin-layer chromatography plates or paper. The concentration can be achieved by direct contact of the carrier with a sufficiently large volume of the sample solution. This can also be done by employing specific chromatographic sample application techniques with a concentrating effect, for example concentration layers from thin-layer chromatography or steep solvent gradients. The substances which have been concentrated and immobilized on the carrier matrix can be tested directly for their biological effect after covering with the active sensor layer.

The effect of the substances in the sample on the sensors in the sensor layer is preferably recorded using imaging methods such as photographic methods, video imaging or else as drawing by hand. Examples of typical reactions of a sensor to a substance are induction or quenching of the emission of light from bioluminescent or chemiluminescent processes, induction or quenching of fluorescent emissions and integral or spectral alteration in the absorption of light. The biological activity is indicated for the positions on the carrier at which a particular substance is located on the basis of the relevant sensor mechanism.

Any number of detection principles—for example bioluminescence, fluorescence, dye formation—allow multiple observation of the detected effect at various times, so that detailed kinetic results on the change in the effect are also obtained. Detection methods via measurement of light emission are preferred to measurements of the spectral alteration in the absorption of light because distinctly greater sensitivities can be achieved therewith and signals are detectable for a considerably shorter time on measurement of light emission.

Analysis of the image data can take place qualitatively in the sense of a yes/no statement about the biological activity and quantitatively to assess levels of effect and the spatial distribution of the activity. This can be done by employing image-processing programmes or else methods of visual comparison, each of which are calibrated with known reference effects.

The substance to be assayed on the carrier may be in unseparated form or, for example, in a form fractionated by thin-layer chromatography or column chromatography. The substance to be assayed on the carrier can also be in a form fractionated by electrophoresis or by another analytical or preparative separation technique. A fractionation of this type is preferably carried out when the sample to be assayed is a mixture of substances.

On chromatographic separation of fractions using a chromatography column, the eluate from the chromatography column is applied either continuously or at intervals to various points on the carrier, for example in the form of a series of spots or by spraying.

The fractionation can be coupled with an investigation of the structure of the individual substances present in the fractions, such that a biological effect detected for a fraction by the sensor layer can be linked to the information about the structure of the individual substance. The information about the structure is obtained from a spectroscopic investigation. The results of the spectroscopic investigation are preferably analysed only after the biological effect of the individual substances has been determined by the sensor layer. The analysis of the results of the spectroscopic investigation preferably takes place only for the individual substances with biological activity.

If the chromatographic separation of the fractions takes place by column chromatography, part of the eluate is applied either continuously or at intervals to various points on the carrier, while another part of the eluate is simultaneously diverted through a mass spectrometer or an NMR spectrometer or an IR spectrometer for spectroscopy (for example using the HPLC/MS, HPLC/NMR, HPLC/IR chromatographic coupling techniques). The eluate from the chromatography column is preferably detected by a UV detector before diverting the portion for spectroscopy.

If the chromatographic separation of the mixture of substances into single substance zones takes place by thin-layer chromatography or electrophoresis on the carrier, it is possible to record, for example from the single substance zones, spectra by matrix assisted laser desorption ionization (MALDI) mass spectroscopy or raman spectroscopy or other spectroscopic methods (UV-VIS, IR) before the carrier is covered with the sensor layer.

The object according to the invention is furthermore achieved by an apparatus consisting of a sensor layer according to the invention which is in contact with the substance to be investigated, and of an imaging system in whose detection zone a part or the whole of the sensor layer is located.

The sensors in the sensor layer preferably indicate their activity by emission or quenching of the emission of light, which is then detected by an appropriate imaging system.

The sensor layer according to the invention, the method according to the invention and the apparatus according to the invention have a number of advantages over the state of the art, in particular for cell-based tests of effects:
1. The sensor layer controls the detection process both by diffusion control and via regulating and assisting additions. Furthermore, the structure of the sensor layer allows uniform coating of different materials (glass, plastic, metal) and surfaces (smooth, rough, porous). Sedimentation of the sensor organisms during the detection process is furthermore avoided.
2. There is often the danger in investigations of bioactive substances that there is interference with the assay if the concentration of the substance to be assayed is too high. This artefact risk is distinctly reduced by the large concentration range detected in the diffusion-controlling sensor layer. The concentration gradient of a substance in the sensor layer permits zones of biological activity below cytotoxic concentrations of substances. This is advantageous for assays of effects of substances whose concentrations in the test are unknown. The effort for determining and standardizing the substance concentrations can be reduced or dispensed with.
3. Mixtures of substances containing cytotoxic or other components interfering with assays of effect can be tested for biological activity with a smaller risk of false-negative results compared with conventional techniques. The diffusion-controlling sensor layer generates different concentration profiles for different substances and thus separates the interfering constituents from those to be assayed in the mixture of substances.
4. If different substances are spatially separate on a carrier, for example in chromatographic zones, there is the danger in the conventional technique that, especially with long incubation times, the substances at different positions on the carrier become mixed. The diffusion-controlling sensor layer prevents excessive diffusion even with long incubation times, so that the zones of active substances on carriers can be identified by their effect even after long incubation times.
5. The method according to the invention permits spatially resolved detection of local variations in the amounts of active substances, for example in parts of plants or animal tissues. It permits high spatial resolutions.
6. It is possible on use of a multisensor layer to investigate the effects of substances on a large number of sensors in a short time. It is easy to construct profiles of effects of active substances.
7. Direct combination of the technique of separating the mixtures of substances on the carrier into fractions and of investigating the fractions by spectroscopy for the structure of the individual substances with detection of the biological activity of the individual substances by the sensor layer allows information to be obtained about the chemical structure of unknown biologically active substances directly from mixtures.

The method according to the invention can be used for investigating biological effects and constructing bioactivity profiles in drug discovery programmes and for mechanistic investigations of the distribution of effects and the release of effects for example in plant or animal tissues.

### FIGURES AND EXAMPLES

The figures show:

FIG. 1 Diagrammatic construction of an apparatus according to the invention.

FIG. 2 Result of a measurement of the spatial distribution of biological activity on a surface with the sensor layer according to the invention.

FIG. 3 Comparison between conventional method and method according to the invention on measurement of a mixture of substances with toxic constituents.

FIG. 4 Experimental construction for coupling chromatographic separation and spectroscopy.

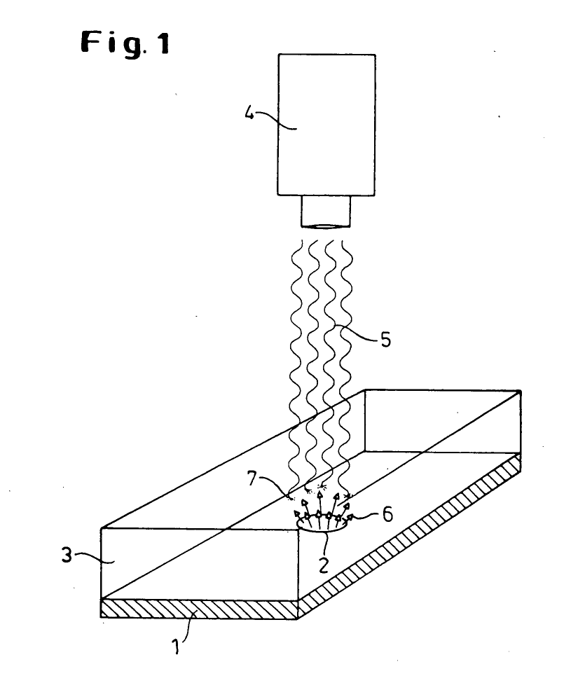

FIG. 1 shows diagrammatically an apparatus according to the invention. An adsorbed substance 2 is present as sample on the carrier 1. The diffusion-controlling sensor layer 3 lies on top of the carrier 1 with the substance 2. An imaging system 4 is located above the sensor layer 3 and detects the optical signal 5. The substance 2 present on the carrier 1 diffuses 6 into the sensor layer 3 and triggers the optical signal 5 on the sensors 7.

### Example 1

Example 1 shows an image of biologically active structures on a solid carrier with the sensor layer according to the invention.

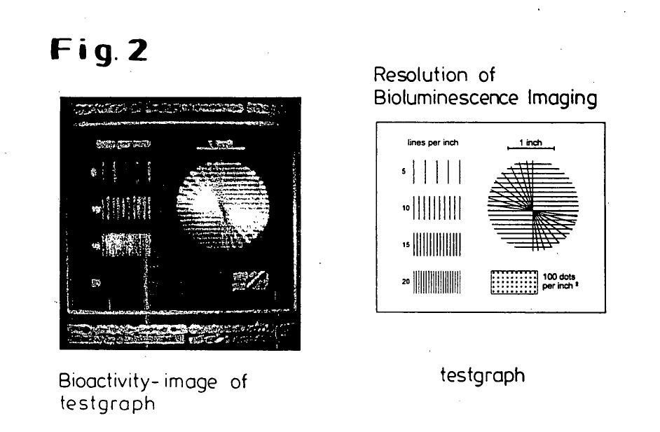

In order to investigate the imaging performance of a sensor layer, the active substance ciprofloxacin was applied by means of an inkjet printer (HP DeskJet 870 Cxi) in the form of a test diagram to a carrier (FIG. 2, right-hand side). The carrier used was a Merck aluminium sheet coated with silica gel 60 (Art. No. 1.05553). To detect the effects, the sensor layer contained reporter gene cells which were produced by genetic manipulation and indicate in an effect-specific manner the biological activity of ciprofloxacin by bioluminescence. The spatial distribution of the effect fixed by the application of ciprofloxacin to the paper carrier was recorded via the bioluminescence induced in the sensor layer using a video imaging system.

The result is depicted in FIG. 2, left-hand side, and shows a graphic resolution of about 20 lpi (lines per inch) for the entire bioluminescence imaging process. This is sufficient to carry out, for example, tests of effects as spot tests with a spot density of more than 25 spots/cm².

Detailed information and the experimental conditions for Example 1 are indicated below:

1. Reporter Gene Cells

The reporter gene cell consists of the Escherichia coli strain SM101 which harbours the recombinant plasmid pEBZ181. This plasmid encodes a fusion between the recA promoter from E. coli and the structural genes of the bacterial luminescence of Vibrio fischeri (lux genes C, D, A, B, E and G). To construct the plasmid, the recA promoter-harbouring Bam H I fragment from the plasmid pUA80 [Barbe J, Fernandez de Henestrosa A R, Calero S, and Gilbert I (1991) Chromogenic Method for rapid isolation of recA-like mutants of Gram-negative bacteria. J. Bacteriol. 173: 404-406] was cloned into a derivative of the vector pEBZ112 [Peitzsch N, Eberz G, and Nies D (1998) Alcaligenes eutrophus as a bacterial chromate sensor. Appl. Environm. Microbiol. 64: 453-458] which harbours, inserted into the Nco I site of the luxG gene, a lacZ cassette [Becker A (1993) Analyse der Succinoglycan-Biosyntheseregion von Rhizobium meliloti 2011: Untersuchungen zur Identifizierung des bakteriellen Infektionssignals in der Symbiose mit Luzeme, doctoral thesis, Bielefeld University, Germany]. Plasmid pEBZ181 was transformed by standard methods [Sambrook J, Fritsch E F, and Maniatis T (1989) Molecular cloning: A laboratory manual (2^{nd} edn), Cold Spring Harbor Laboratory Press] into the Escherichia coli strain SMIOI (obtained from E. coli Genetic Stock Center, Yale University, New Haven, USA) and employed for bioimaging experiments.

Treatment of bacteria with antibacterial agents such as 4-quinolonecarboxylic acids leads to induction of the so-called SOS repair mechanism [Walker C G (1984) Mutagenesis and inducible responses to deoxyribonucleic acid damage in Escherichia coli. Microbiol. Rev. 48: 60-93; Phillips I, Culabras E, Moreno F, and Baquero F (1987) Induction of the SOS response by new 4-quinolones. J. Antimicrob. Chemother. 20: 631-638; Piddock L J V, and Wise R (1987) Induction of the SOS response in Escherichia coli by 4-quinone antimicrobial agents. FEMS Microbiol. Lett. 41: 289-294]. The RecA protein is a main regulator of this repair mechanism, and SOS induction leads to enhanced expression of the recA gene. Measurement of the synthesis of the RecA protein [Little J W and Mount D W (1982) The SOS regulatory system of Escherichia coli. Cell 29: 11-22; Witkin E M (1976) Ultraviolet mutagenesis and inducible DNA repair in Escherichia coli. Bacteriol. Rev. 40: 864-907] or of recA-reporter gene fusions [Nunoshiba T and Nishioka H (1991) Rec-lac test for detecting SOS-inducing activity of environmental genotoxic substances. Mutation Res. 254:71-77] is one possible way of measuring SOS induction and consequently the effect of 4-quinolonecarboxylic acids. Since the strain E. coli SM101 (pEBZ181) harbours a plasmid with a recA-lux reporter gene fusion, it is suitable for detecting 4-quinolonecarboxylic acids or other SOS-inducing compounds. The presence of such compounds leads to enhanced expression of the luciferase gene and thus to a stimulation of bioluminescence.

2. Sensor Layer Composition

The coating composition was as follows:
32 ml of 1% agarose (agarose MP Boehringer Mannheim GmbH Art. No. 1388983); this low-melting agarose allows temperature-sensitive reporter gene cells to be suspended at temperatures below 40° C. without damage.
4 ml of LB medium 200 g/l (GIBCO BRL Art. No. 12780-052); the addition of LB medium serves to maintain the vital functions of the reporter gene cells during the incubation period.
4 ml of bacterial suspension in LB medium 20 g/l; this resulted in an optical density of 1.2 at a wavelength of 660 nm; the cell density, that is to say the density of the sensors in the sensor layer, is controlled via the volume of bacterial suspension added in order to optimize the signal/noise ratio and the spatial resolution.
10 ml of water; this addition controls the viscosity of the casting composition and the mechanical stability of the sensor layer after curing.
   3. Test Diagram

The test diagram was produced using Corel Draw (Version 8). The line thickness for the array of lines is 0.1 mm. The dot matrix contains 50 square dots (edge length 1 mm) in ½ inch². The dots were printed with a colour saturation of 50%; all the other elements of the test diagram were printed with 100% colour saturation.

4. Ciprofloxacin Application to Aluminium Sheet Coated with Silica Gel

42 ml of a solution of 120 mg of ciprofloxacin hydrochloride monohydrate in water were placed in a previously emptied ink cartridge of the HP DeskJet 870 Cxi inkjet printer. The test diagram described under 3. (FIG. 2, right-hand side) was printed on the silica gel layer of the aluminium sheet (Merck Art. No. 1.05553) by the inkjet printer.

5. Application of the Sensor Layer and Incubation

The aluminium sheet was dried under a stream of nitrogen, inserted into an appropriate stainless steel frame with raised rim and positioned horizontally on a levelling stage, and the sensor layer composition obtained under 2. was poured on uniformly to result in a layer thickness of 2 mm. After curing of the sensor layer at room temperature, the coated carrier was incubated at 28° C. for 60 minutes.

6. Video Imaging

The carrier incubated according to 5. was measured using a video imaging system (“Molecular Light Imager NightOWL” from EG&G Berthold) in accordance with the operating instructions. The picture-taking period was 60 s, and the camera position was optimized for an image format of 10×20 cm. To display the results, the resulting image data were converted into TIFF files and then formatted with suitable graphics programmes (Corel Draw Version 8, Corel Photo Paint Version 8 or Photoshop Version 4.0), captioned and printed out by a laser printer (HP LaserJet 5). FIG. 2 shows the bioluminescence image of the effect of the ciprofloxacin printed onto the silica gel/aluminium sheet, together with the original test diagram.

### Example 2

Example 2 shows an application of the sensor layer for activity assays in the presence of cytotoxic substances and a comparison with an analogous microtitre plate assay.

In contrast to conventional microtitre plate formats, the diffusion-controlling sensor layer can be employed for assays of mixtures of substances which contain interfering components, since the interfering components are removed by diffusion and adsorption processes in situ so that the risk of false-negative results is reduced. The superiority of the sensor layer compared with microtitre plate formats can be demonstrated by the example of bioactivity assays using the SOS reporter gene system (see Example 1).

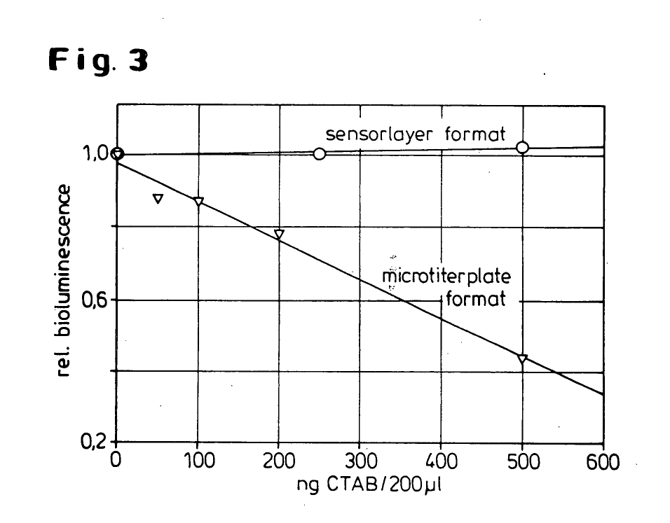

In both assay formats ciprofloxacin was used for effect-specific induction of the SOS response. The bioluminescence stimulated in the reporter gene system used in this case provided the read-out signal which was recorded by video imaging. In order to test the tolerance of the two bioassay formats to interfering substances, assays were carried out in the presence of cytotoxic cetyltrimethylammonium bromide (CTAB). This revealed that the relative bioluminescence, as a measure of the selective stimulation by ciprofloxacin, remained constant for the sensor layer format up to CTAB concentrations of 500 ng/200 μl, whereas the microtitre plate format showed a reduction of more than 50% in bioluminescence in this region (FIG. 3).

Detailed information and the experimental conditions for Example 2 are given below:

1. SOS Reporter Gene System

The SOS reporter gene system used was the same as in Example 1.

2. Microtitre Plate Assays

Five assay solutions were made up in a 96-well microtitre plate (Dynatech Microlite). A volume of 200 μl contained in each case:

94 μl of 2% LB medium (GIBCO BRL Art. No. 12780-052)

16 μl of reporter gene cell suspension (OD: 1.2 at 660 nm) in 2% LB medium

90 μl of solution of substances 1.-5. in water

1. 25 ng of ciprofloxacin hydrochloride monohydrate

2. 25 ng of ciprofloxacin hydrochloride monohydrate+50 ng of CTAB

3. 25 ng of ciprofloxacin hydrochloride monohydrate+100 ng of CTAB

4. 25 ng of ciprofloxacin hydrochloride monohydrate+200 ng of CTAB

5. 25 ng of ciprofloxacin hydrochloride monohydrate+500 ng of CTAB

The microtitre plate was incubated at 28° C. for 60 min and then measured using the video imaging system “Molecular Light Imager NightOWL” from EG&G Berthold. The average grey values for each of the individual wells were determined. The relative bioluminescence was found by dividing these grey values by the value for the CTAB-free reference solution. The average relative bioluminescence intensity was determined as a function of the CTAB concentration for two series of measurements:

| CTAB | |
|---|---|
| [ng/200 μl] | Rel. bioluminescence |
| 0 | 1.00 |
| 50 | 0.88 |
| 100 | 0.87 |
| 200 | 0.78 |
| 500 | 0.44 |

For comparison, the results are compared in the figure below with the results for the sensor layer format.

3. Activity Measurements with the Sensor Layer

To detect the effect of ciprofloxacin using the sensor layer, the following assay solutions were applied to a thin-layer chromatography plate (Merck Art. No. 1.15445 Si 60 F₂₅₄ₛ) using disposable micropipettes:
1. 1 μl of aqueous solution of 10 ng of ciprofloxacin hydrochloride monohydrate
2. 1 μl of aqueous solution of 10 ng of ciprofloxacin hydrochloride monohydrate+50 ng of CTAB
3. 1 μl of aqueous solution of 10 ng of ciprofloxacin hydrochloride monohydrate+100 ng of CTAB

The TLC plates were dried under a stream of nitrogen and underwent planar coating on a levelling stage with a layer thickness of about 2 mm using a suspension of the luminescent sensor bacteria (see Example 1) in 0.6% agarose. The TLC plates were then incubated at 28° C. for 60 min. The induced bioluminescence was subsequently detected by imaging using the high-resolution CCD low light imaging system “Molecular Light Imager NightOWL” from EG&G Berthold. Imaging conditions: picture-taking time 60 s; camera position optimized for 10×20 cm TLC plate format. The average grey values were determined for the luminescent spots. For comparison with the results described above for the microtitre plate format, the analogous concentrations were calculated from the amounts of substance supplied to the thin-layer plate. For this estimate it was assumed that the substances diffuse in the sensor layer volume above the substance spots (spot diameter 5 mm, layer thickness 2 mm). Two series of measurements provided the following relative bioluminescence intensities (averages) as a function of the CTAB concentration:

| CTAB | |
|---|---|
| [ng/200 μl] | Rel. bioluminescence |
| 0 | 1.000 |
| 250 | 1.002 |
| 500 | 1.023 |

4. Comparison of the Results

Comparison of the measurements with the two formats shows that the bioactivity measurement was not impaired by the addition of CTAB in the case of the sensor layer, whereas there was a distinct fall (about 40% of the initial value) in the bioluminescence with the microtitre plate format. These results are compared in the graph in FIG. 3.

### Example 3

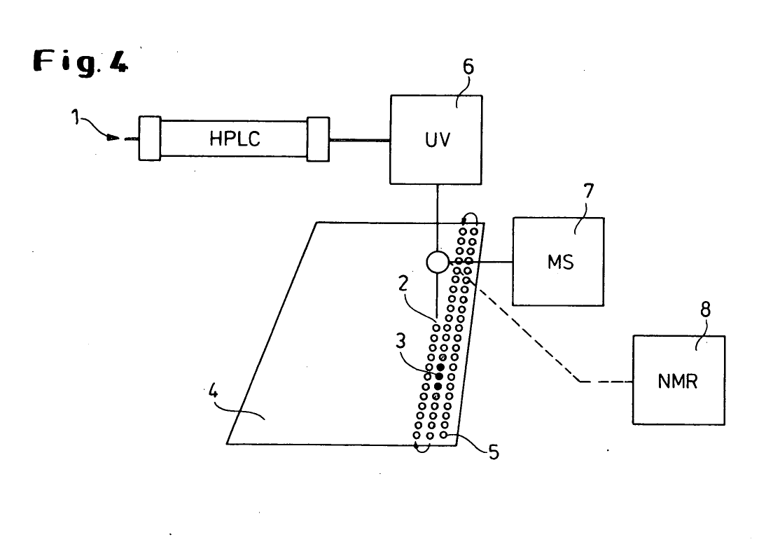

FIG. 4 is a diagrammatic representation of how a mixture of substances is fractionated by column chromatography and, at the same time, spectroscopic data on the individual fractions are obtained.

Sample 1 is subjected to column chromatography (HPLC). The eluate from the chromatography column is detected by a UV detector 6. Part of the eluate is applied 2 by spraying or spotting to a carrier 4 via a split system. Beginning at a start position 5, the column chromatography eluate is placed in the form of an array on the carrier so that all the substances from the column chromatography run are deposited spatially separate thereon. Another part of the eluate is at the same time diverted into a mass spectrometer 7 or an NMR spectrometer 8 for spectroscopy therein (coupled HPLC/MS or HPLC/NMR). After all the spots have been applied to the carrier, the carrier is, where appropriate after drying, covered with the sensor layer. The sensor layer indicates the positions of the biologically active fractions 3. The mass spectra or NMR data are assigned unambiguously to the individual spots by correlation with the column chromatography elution times. This means that detailed structural information on the active substances is available immediately after completion of the test of biological effects.

1. A method for detecting the biological effect of a substance, comprising the steps of:
a.) placing a sample to be assayed onto a surface of, or into a carrier;
b.) covering the carrier with a sensor layer, the sensor layer comprising a diffusion-controlling matrix and sensors suspended therein; and
c.) determining the effect of the substance present in the sample on the sensors in the sensor layer.

2. The method according to claim 1, wherein sensors suspended in the sensor layer are reporter gene cells, which are present in a concentration of 2 to 8 ml of reporter gene cell suspension per 50 ml of sensor layer composition.

3. The method according to claim 2, wherein reporter gene cells are present in a concentration of 3 to 5 ml of reporter gene cell suspension per 50 ml of sensor layer composition.

4. The method according to claim 1, wherein the reporter gene cells are bio-luminescent.

5. The method according to claim 1, wherein the sensor layer is used as a carrier.

6. The method according to claim 1, wherein the step of determining the effect of the substance present in the sample on the sensors is preceded by an incubation step in which the carrier covered with the sensor layer is stored under defined conditions, for a preset time.

7. The method according to claims 1, wherein the effect of the substance on the sensors consists of location-dependent induction or quenching of the emission of light from bioluminescent processes.

8. The method according to claim 1, wherein the substance in the sample is concentrated by specific or nonspecific adsorption onto a suitable carrier material before the carrier material is brought into contact with the sensor layer.

9. The method according to claim 1, wherein the sample is a mixture of substances.

10. The method according to claim 9, wherein the mixture is fractionated by chromatography, electrophoresis or using another analytical or preparative separation technique before it is brought into contact with the sensor layer.

11. The method according to claim 10, wherein the fractionation takes place in a chromatography column, and the eluate is applied either continuously or at intervals to various points on the carrier.

12. The method according to claim 10, wherein the fractionation takes place by thin-layer chromatography or electrophoresis on the carrier, which is covered with the sensor layer.

13. The method according to claim 9, wherein the determination of the effect of the individual substances in the mixture of substances is linked to a detection of the structure of the individual substances by fractionation of the mixture by chromatography or electrophoresis or with other analytical or preparative separation techniques, and investigation by spectroscopy of each fraction before the individual substances are brought into contact with the sensor layer.

14. The method according to claim 13, further comprising the step of analyzing structure data from the spectroscopic investigation only for the fractions for which a biological effect is detected by the sensor layer.

15. The method according to claim 13, wherein the fractionation of the mixture of substances takes place in a chromatography column, and part of the eluate is continuously applied to various points on the carrier, while another part of the eluate is simultaneously diverted through a mass spectrometer, a NMR spectrometer or an IR spectrometer for spectroscopy.

16. The method according to claim 15, wherein the eluate from the chromatography column is detected by a UV detector before diverting the portion for spectroscopy.

17. The method according to claim 13, wherein the fractionation of the mixture of substances takes place by thin-layer chromatography or electrophoresis on the carrier, so that each fraction is separated into single substance zones.

18. The method according to claim 17, wherein each single substance zone is analyzed by MALDI mass spectroscopy, raman spectroscopy, UV-VIS spectroscopy or IR spectroscopy before the covering step.

19. A method for detecting the biological effect of a substance, comprising the steps of:
a.) providing a carrier having a sample to be assayed;
b.) covering the carrier with a sensor layer, the sensor layer comprising a diffusion-controlling matrix and sensors suspended therein; and
c.) determining the effect of the substance present in the sample on the sensors in the sensor layer.

20. The method according to claim 19, wherein sensors suspended in the sensor layer are reporter gene cells, which are present in a concentration of 2 to 8 ml of reporter gene cell suspension per 50 ml of sensor layer composition.

21. The method according to claim 20, wherein reporter gene cells are present in a concentration of 3 to 5 ml of reporter gene cell suspension per 50 ml of sensor layer composition.

22. The method according to claim 19, wherein the reporter gene cells are bio-luminescent.

23. The method according to claim 19, wherein the carrier serves as a sensor layer.
